(19) United States
(12) Patent Application Publication
(10) Pub. No.: US 2026/0191645 A1
(43) Pub. Date: Jul. 9, 2026

(54) PARAVALVULAR LEAKAGE SEALING MEMBER FOR PROSTHETIC VALVE

(71) Applicant: Edwards Lifesciences Corporation, Irvine, CA (US)

(72) Inventors: Jocelyn Chau, Buena Park, CA (US); Tram Ngoc Nguyen, Santa Ana, CA (US)

(21) Appl. No.: 19/551,497

(22) Filed: Feb. 26, 2026

Related U.S. Application Data
(63) Continuation of application No. PCT/US2024/044920, filed on Aug. 31, 2024.
(60) Provisional application No. 63/580,309, filed on Sep. 1, 2023.

(51) Int. Cl. A61F 2/24

(52) U.S. Cl. CPC A61F 2/2433; A61F 2/2418; A61F 2230/0006; A61F 2250/0069

(57) ABSTRACT

An implantable prosthetic valve can include a radially expandable and compressible annular frame comprising an inflow end portion and an outflow end portion, and a valvular structure comprising a plurality of leaflets arranged within the frame. The prosthetic valve can further include a sealing member having a first end portion and a second end portion, the first end portion being coupled to an inflow edge of the frame, the sealing member being movable between a delivery configuration wherein the first end portion extends proximally from the frame and a deployed configuration wherein the first end portion is everted over the inflow end of the frame such that the second end portion forms a flanged portion.

### CROSS REFERENCE TO RELATED APPLICATION

This application is a continuation of PCT Patent Application No. PCT/US2024/044920, filed on Aug. 31, 2024, which application claims the benefit of U.S. Provisional Patent Application No. 63/580,309, filed on Sep. 1, 2023, each of these applications being incorporated by reference herein in its entirety.

### FIELD

The present disclosure relates to prosthetic medical devices and delivery apparatuses for preventing or mitigating paravalvular leakage for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic heart valves, delivery apparatus, and methods for implanting prosthetic heart valves. The disclosed prosthetic heart valves, delivery apparatus, and methods can, for example, provide a sealing member to prevent or mitigate paravalvular leakage (PVL) without increasing the crimp profile of the prosthetic valves during delivery. The sealing member can be disposed over a portion of the delivery apparatus such that is can be actuated by the delivery apparatus to position the sealing member in a deployed configuration. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage. In some examples, the sealing member can extend proximally from an inflow end portion of the prosthetic heart valve over a portion of the delivery apparatus when the prosthetic heart valve is mounted on the delivery apparatus.

In some examples, a prosthetic heart valve can comprise an outer skirt.

In some examples, a prosthetic heart valve can comprise a radially expandable and compressible annular frame comprising an inflow end portion and an outflow end portion, a valvular structure comprising a plurality of leaflets arranged within the frame, and a sealing member having a first end portion and a second end portion, the first end portion being coupled to an inflow edge of the frame, the sealing member being movable between a delivery configuration wherein the first end portion extends proximally from the frame and a deployed configuration wherein the first end portion is everted over the inflow end of the frame such that the second end portion forms a flanged portion.

In some examples, an assembly can comprise a guide catheter including a guide catheter shaft, a prosthetic valve delivery apparatus comprising a handle, a delivery shaft extending from a distal end portion of the handle and comprising a delivery shaft lumen, and an inflatable member coupled to a distal end portion of the delivery shaft, and a radially expandable and compressible implantable prosthetic device disposed over the inflatable member. The prosthetic device can comprise an annular frame comprising an inflow end portion and an outflow end portion, a valvular structure comprising a plurality of leaflets arranged within the frame, and a sealing member having a first end portion and a second end portion, the first end portion being coupled to an inflow edge of the frame, the sealing member being movable between a delivery configuration and a deployed configuration. When the sealing member is in the delivery configuration the sealing member can extend proximally over the delivery shaft of the prosthetic valve delivery apparatus.

In some examples, a prosthetic heart valve and/or an assembly can comprise one or more of the components recited in Examples 1-27 below.

In some examples, a method of delivering an implantable prosthetic valve to a native valve of a heart can comprise advancing a distal end portion of a delivery apparatus through a guide catheter shaft to a selected implantation site, the distal end portion of the delivery apparatus comprising a prosthetic valve disposed over an inflatable member, the prosthetic valve comprising a radially expandable and compressible annular frame, a valvular structure comprising a plurality of leaflets arranged within the frame, and a sealing member having a first end portion and a second end portion, the first end portion being coupled to an inflow edge of the frame and the second end portion being disposed over a shaft of the delivery apparatus. The method can further comprise using a distal end portion of the guide catheter shaft to partially evert the sealing member over an inflow end portion of the prosthetic valve such that the first end portion of the sealing member is disposed adjacent an outer surface of the prosthetic valve and such that the second end portion of the sealing member forms a flanged portion extending laterally outwardly from an outer surface of the prosthetic valve, positioning the prosthetic valve at the selected implantation site, and radially expanding the prosthetic valve to seat the prosthetic valve at the selected implantation site.

In some examples, a method can comprise one or more of the acts recited in Examples 28-32 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 schematically illustrates a stage in an example mitral valve replacement procedure where a guide catheter and a guidewire are inserted into a blood vessel of a patient and navigated through the blood vessel and into a heart of the patient, towards a native mitral valve of the heart.

FIG. 2A schematically illustrates another stage in the example mitral valve replacement procedure where a docking device delivery apparatus extending through the guide catheter is implanting a docking device for a prosthetic heart valve at the native mitral valve.

FIG. 2B schematically illustrates another stage in the example mitral valve replacement procedure where the docking device of FIG. 2A is fully implanted at the native mitral valve of the patient and the docking device delivery apparatus has been removed from the patient.

FIG. 3A schematically illustrates another stage in the example mitral valve replacement procedure where a prosthetic heart valve delivery apparatus extending through the guide catheter is implanting a prosthetic heart valve in the implanted docking device at the native mitral valve.

FIG. 3B schematically illustrates another stage in the example mitral valve replacement procedure where the prosthetic heart valve is fully implanted within the docking device at the native mitral valve and the prosthetic heart valve delivery apparatus has been removed from the patient.

FIG. 4 schematically illustrates another stage in the example mitral valve replacement procedure where the guide catheter and the guidewire have been removed from the patient.

FIG. 5 is a perspective view of a prosthetic heart valve delivery apparatus, according to one example, with the prosthetic heart valve shown in the radially compressed configuration.

FIG. 6 is a perspective view of a portion of the prosthetic heart valve and delivery apparatus of FIG. 5, with the prosthetic heart valve shown in the radially expanded configuration.

FIG. 7 is a perspective view of a prosthetic heart valve, according to one example.

FIG. 8 is a perspective view of the prosthetic heart valve of FIG. 7 seated within an exemplary docking device, according to one example.

FIGS. 9A-9D are perspective views of a method of everting the sealing member of the prosthetic heart valve of FIG. 7 using a guide catheter, according to one example.

FIG. 10 is a schematic view of a prosthetic heart valve seated within a docking device in a native mitral valve, according to one example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Disclosed herein are examples of an implantable prosthetic medical device (such as a docking device used in conjunction with a prosthetic heart valve) and a delivery system that can be used to navigate a subject's vasculature to deliver the prosthetic medical device, tools, agents, or other therapy to a target implantation site within the body of the subject. In connection therewith, the implantable medical devices and/or methods described herein can, in some examples, comprise a sealing member configured to prevent or mitigate paravalvular leakage at the implantation site. Disclosed herein are exemplary devices and/or methods that can, among other things, advantageously allow for the addition of a sealing member without increasing the overall crimp profile of the implantable medical device during delivery.

### Examples of the Disclosed Technology

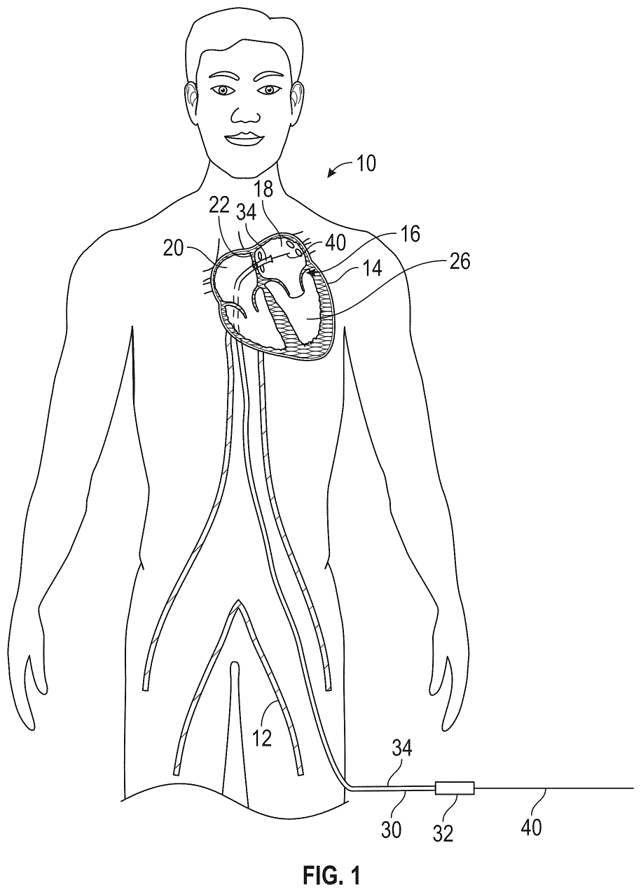

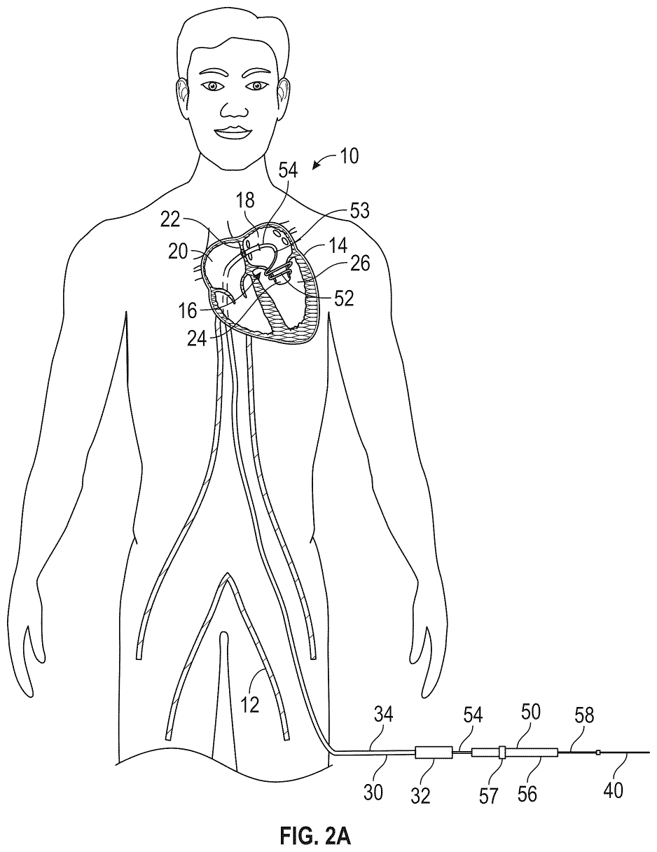

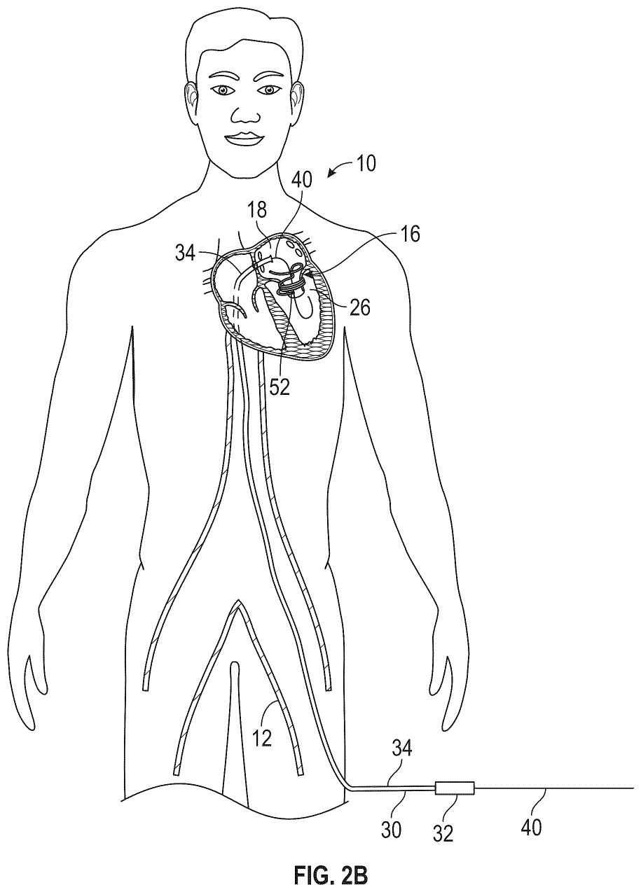

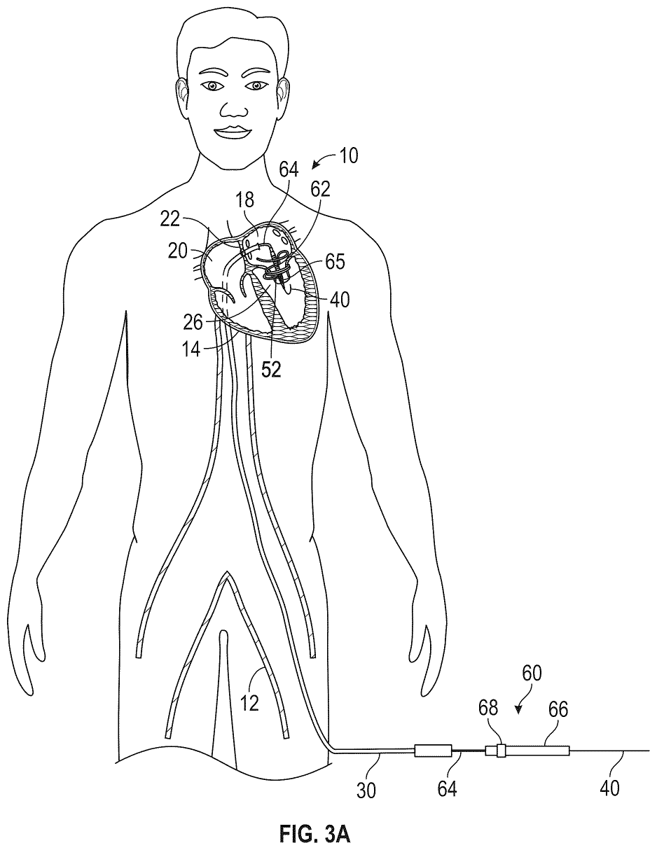

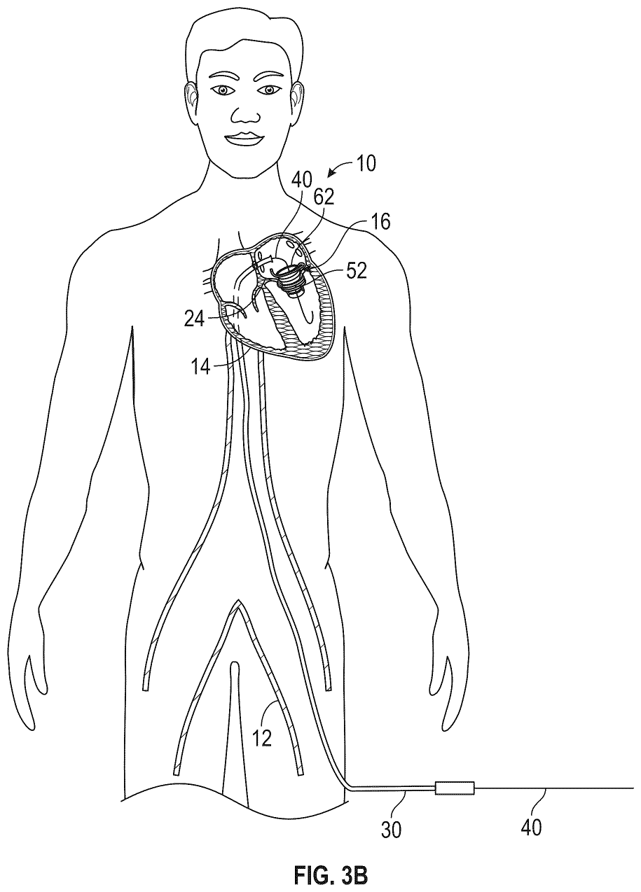

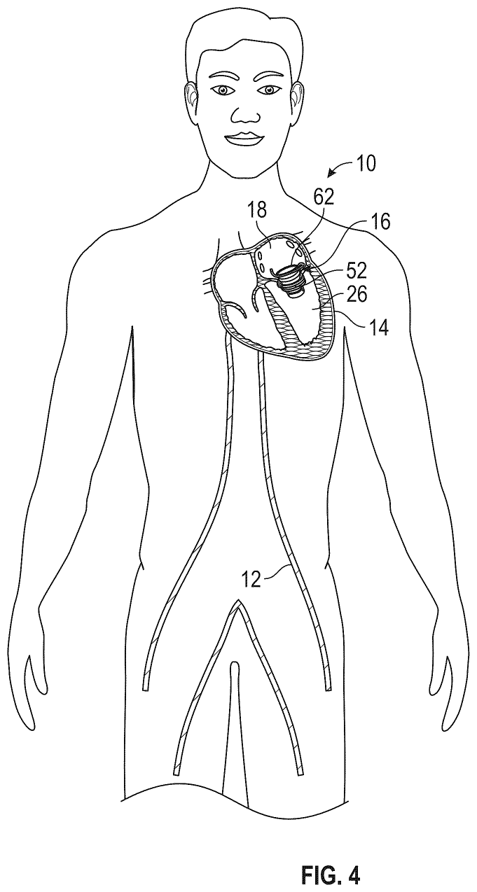

FIGS. 1-4 depict an example of a transcatheter heart valve replacement procedure (such as a mitral valve replacement procedure) which utilizes a docking device 52 and a prosthetic heart valve 62 (FIG. 3B), according to one example. During the procedure, a user first creates a pathway to a patient's native heart valve using a guide catheter 30 (FIG. 1). The user then delivers and implants the docking device 52 at the patient's native heart valve through the guide catheter 30 using a first delivery apparatus configured as a docking device delivery apparatus 50 (FIG. 2A) and then removes the first delivery apparatus 50 from the patient 10 after implanting the docking device 52 (FIG. 2B). The user then implants the prosthetic heart valve 62 within the implanted docking device 52 using a second delivery apparatus configured as a prosthetic valve delivery apparatus 60 (FIG. 3A). Thereafter, the user removes the second delivery apparatus 60 from the patient 10 (FIG. 3B), as well as the guide catheter 30 (FIG. 4).

FIG. 1 depicts a stage in a mitral valve replacement procedure, according to one example, where the guide catheter 30 and a guidewire 40 are inserted into a blood vessel 12 of a patient 10 and navigated through the blood vessel 12, into a heart 14 of the patient 10, and toward the native mitral valve 16. Together, the guide catheter 30 and the guidewire 40 can provide a path for the first delivery apparatus 50 (also referred to as the “docking device delivery apparatus”) and the second delivery apparatus 60 (also referred to as the “prosthetic valve delivery apparatus”) to be navigated through and along, to the implantation site (e.g., the native mitral valve 16 or native mitral valve annulus). As shown, the heart 14 is illustrated schematically. For example, the anterior leaflet and chordae of the native mitral valve 16 are omitted for illustration purposes, such that only a portion of the posterior leaflet of the native mitral valve 16 is illustrated.

Initially, the user may first make an incision in the patient's body to access the blood vessel 12. For example, in the example illustrated in FIG. 1, the user may make an incision in the patient's groin to access a femoral vein. Thus, in such examples, the blood vessel 12 may be a femoral vein.

After making the incision at the blood vessel 12, the user may insert the guide catheter 30, the guidewire 40, and/or additional devices (such as an introducer device or transseptal puncture device) through the incision and into the blood vessel 12. The guide catheter 30 (which can also be referred to as an “introducer device,” “introducer,” or “guide sheath”) is configured to facilitate the percutaneous introduction of various implant delivery devices (such as the delivery apparatus 50 and the prosthetic valve delivery apparatus 60) into and through the blood vessel 12 and may extend through the blood vessel 12 and into the heart 14 but may stop short of the native mitral valve 16. The guide catheter 30 can comprise a handle 32 and a shaft 34 (which may also be referred to as a catheter shaft 34) extending distally from the handle 32. The shaft 34 can extend through the blood vessel 12 and into the heart 14 while the handle 32 remains outside the body of the patient 10 and can be operated by the user in order to manipulate the shaft 34 (FIG. 1).

The guidewire 40 is configured to guide the delivery apparatuses (such as the guide catheter 30, the delivery apparatus 50, the prosthetic valve delivery apparatus 60, additional catheters, or the like) and their associated devices (such as docking device, prosthetic heart valve, and the like) to the implantation site within the heart 14, and thus may extend all the way through the blood vessel 12 and into a left atrium 18 of the heart 14 (FIG. 1) and in some examples, through the native mitral valve 16 and into a left ventricle 26 of the heart 14.

In some instances, a transseptal puncture device or catheter can be used to initially access the left atrium 18, prior to inserting the guidewire 40 and the guide catheter 30. For example, after making the incision to the blood vessel 12, the user may insert a transseptal puncture device through the incision and into the blood vessel 12. The user may guide the transseptal puncture device through the blood vessel 12 and into the heart 14 (such as through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the blood vessel 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the blood vessel 12 and advance the guide catheter 30 into the left atrium 18 over the guidewire 40 (FIG. 1).

In some instances, an introducer device can be inserted through a lumen of the guide catheter 30 prior to inserting the guide catheter 30 into the blood vessel 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts another stage in the example mitral valve replacement procedure where a docking device 52 is being implanted at the native mitral valve 16 of the heart 14 of the patient 10 using a delivery apparatus 50 (which may also be referred to as a “first delivery apparatus,” an “implant catheter,” a “dock delivery system,” a “docking device delivery apparatus,” and/or a “docking device delivery device”).

In general, the delivery apparatus 50 comprises a delivery shaft 54 (which may also be referred to as a “dock delivery system shaft”), a handle 56 (which may also be referred to as a “dock delivery system handle”), and a pusher assembly 58. The delivery shaft 54 is configured to be advanced through the patient's vasculature (blood vessel 12) and to the implantation site (such as native mitral valve 16) by the user and may be configured to retain the docking device 52 in a distal end portion 53 of the delivery shaft 54. In some examples, the distal end portion 53 of the delivery shaft 54 retains the docking device 52 therein in a straightened delivery configuration.

The handle 56 of the delivery apparatus 50 is configured to be gripped and/or otherwise held by the user, outside the body of the patient 10, to advance the delivery shaft 54 through the patient's vasculature (such as the blood vessel 12).

In some examples, the handle 56 can comprise one or more articulation members 57 (or rotatable knobs) that are configured to aid in navigating the delivery shaft 54 through the blood vessel 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the blood vessel 12 and within the heart 14.

The pusher assembly 58 can be configured to deploy and/or implant the docking device 52 at the implantation site (such as the native mitral valve 16). For example, the pusher assembly 58 is configured to be adjusted by the user to push the docking device 52 out of the distal end portion 53 of the delivery shaft 54. A shaft (which may also be referred to as a “pusher shaft”) of the pusher assembly 58 can extend through the delivery shaft 54 and can be disposed adjacent to the docking device 52 within the delivery shaft 54. In some examples, the docking device 52 can be releasably coupled to the shaft of the pusher assembly 58 via a connection mechanism of the delivery apparatus 50 such that the docking device 52 can be released after being deployed at the native mitral valve 16.

Further details of the docking device delivery apparatus and its variants can be found, at least, in International Publication Nos. WO2020/247907 and WO2023/205076 and WO2024/091366, which are incorporated by reference herein in their entirety.

Referring again to FIG. 2A, after the guide catheter 30 is positioned within the left atrium 18, the user may insert the delivery apparatus 50 (such as the delivery shaft 54) into the patient 10 by advancing the delivery shaft 54 of the delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the delivery apparatus 50 through the blood vessel 12 along the guidewire 40 until the delivery shaft 54 reaches the left atrium 18, as illustrated in FIG. 2A. Specifically, the user may advance the delivery shaft 54 of the delivery apparatus 50 by gripping and exerting a force on (for example, by pushing) the handle 56 of the delivery apparatus 50 toward the patient 10. While advancing the delivery shaft 54 through the blood vessel 12 and the heart 14, the user may adjust the one or more articulation members 57 of the handle 56 to navigate the various turns, corners, constrictions, and/or other obstacles in the blood vessel 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (such as the articulation members 57). In some examples, the user can fine tune the positioning of the distal end portion 53 of the delivery shaft 54 using the methods described in International App. No. PCT/US2024/018313, which is incorporated by reference herein in its entirety. After the delivery shaft 54 is positioned, the user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (such as the portion of the docking device 52 shown in FIG. 2A that is configured to be positioned within the left ventricle 26 and/or on the ventricular side of the native mitral valve 16), the user may then deploy the remaining portion of the docking device 52 (such as an atrial portion of the docking device 52) from the delivery shaft 54 within the left atrium 18 by retracting the delivery shaft 54 away from the posteromedial commissure of the native mitral valve 16.

After deploying and implanting the docking device 52 at the native mitral valve 16, the user may disconnect the delivery apparatus 50 from the docking device 52. Once the docking device 52 is disconnected from the delivery apparatus 50, the user may retract the delivery apparatus 50 out of the blood vessel 12 and away from the patient 10 so that the user can deliver and implant a prosthetic heart valve 62 within the implanted docking device 52 at the native mitral valve 16.

FIG. 2B depicts this stage in the mitral valve replacement procedure, where the docking device 52 has been fully deployed and implanted at the native mitral valve 16 and the delivery apparatus 50 (including the delivery shaft 54) has been removed from the patient 10 such that only the guidewire 40 and the guide catheter 30 remain inside the patient 10. In some examples, after removing the delivery apparatus 50, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of turns (or coils) that wrap around the leaflets 24 of the native mitral valve 16 (within the left ventricle 26). The implanted docking device 52 has a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts another stage in the mitral valve replacement procedure where the user is delivering and/or implanting a prosthetic heart valve 62 (which can also be referred to herein as a “transcatheter heart valve” or “THV” for short, “replacement heart valve,” and/or “prosthetic mitral valve”) within the docking device 52 using a prosthetic valve delivery apparatus 60.

As shown in FIG. 3A, the prosthetic valve delivery apparatus 60 can comprise a delivery shaft 64 and a handle 66, the delivery shaft 64 extending distally from the handle 66. The delivery shaft 64 is configured to extend into the patient's vasculature to deliver, implant, expand, and/or otherwise deploy the prosthetic heart valve 62 within the docking device 52 at the native mitral valve 16. The handle 66 is configured to be gripped and/or otherwise held by the user to advance the delivery shaft 64 through the patient's vasculature.

In some examples, the handle 66 can comprise one or more articulation members 68 that are configured to aid in navigating the delivery shaft 64 through the blood vessel 12 and the heart 14. Specifically, the articulation member(s) 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (such as the expansion mechanism) configured to radially expand the prosthetic heart valve 62.

As shown in FIG. 3A, the prosthetic heart valve 62 is mounted around the expansion mechanism 65 (the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (the delivery shaft 64) into the patient 10 through the guide catheter 30 and over the guidewire 40. The user can continue to advance the prosthetic valve delivery apparatus 60 along the guidewire 40 (through the blood vessel 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, by pushing) the handle 66. While advancing the delivery shaft 64 through the blood vessel 12 and the heart 14, the user can adjust the one or more articulation members 68 of the handle 66 to navigate the various turns, corners, constrictions, and/or other obstacles in the blood vessel 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, by inflating the inflatable balloon), thereby radially expanding the prosthetic heart valve 62 within the docking device 52.

FIG. 3B shows another stage in the mitral valve replacement procedure where the prosthetic heart valve 62 in its radially expanded configuration and implanted within the docking device 52 in the native mitral valve 16. As shown in FIG. 3B, the prosthetic heart valve 62 is received and retained within the docking device 52. Thus, the docking device 52 aids in anchoring the prosthetic heart valve 62 within the native mitral valve 16. The docking device 52 can enable better sealing between the prosthetic heart valve 62 and the leaflets 24 of the native mitral valve 16 to reduce paravalvular leakage around the prosthetic heart valve 62.

As also shown in FIG. 3B, after the prosthetic heart valve 62 has been fully deployed and implanted within the docking device 52 at the native mitral valve 16, the prosthetic valve delivery apparatus 60 (including the delivery shaft 64) is removed from the patient 10 such that only the guidewire 40 and the guide catheter 30 remain inside the patient 10.

FIG. 4 depicts another stage in the mitral valve replacement procedure, where the guidewire 40 and the guide catheter 30 have been removed from the patient 10.

Although FIGS. 1-4 specifically depict a mitral valve replacement procedure, it should be appreciated that the same and/or similar procedure may be utilized to replace other heart valves (such as tricuspid, pulmonary, and/or aortic valves). Further, the same and/or similar delivery apparatuses (such as the delivery apparatus 50, prosthetic valve delivery apparatus 60, guide catheter 30, and/or guidewire 40), docking devices (such as the docking device 52), replacement heart valves (such as the prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned/disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

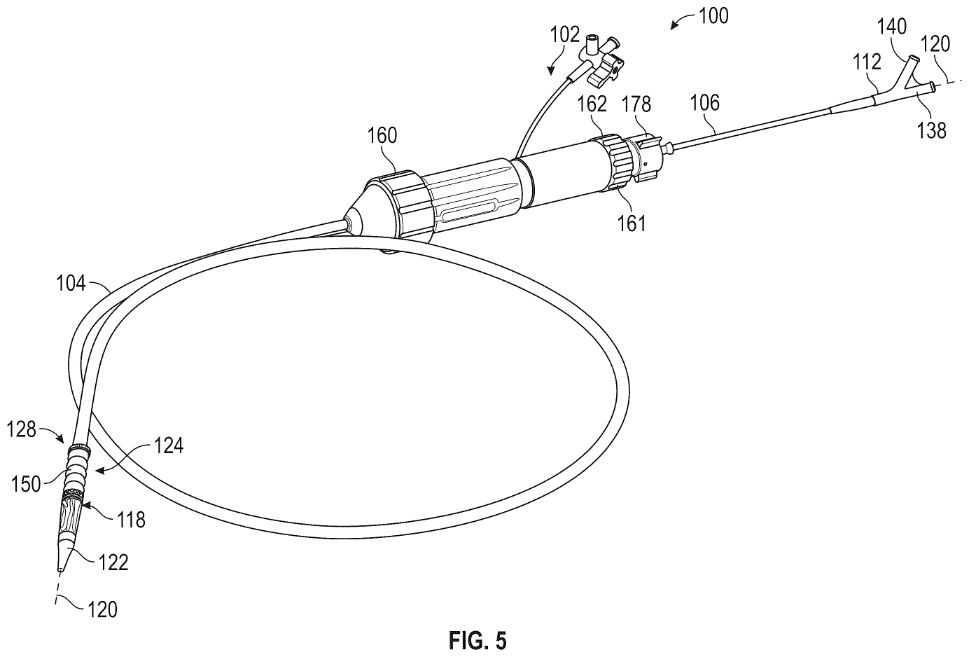

FIG. 5 illustrates an exemplary prosthetic valve delivery apparatus 100 (which can also be referred to here as an “implant catheter” and/or a “prosthetic heart valve delivery apparatus”) that can be used to implant an expandable prosthetic heart valve, according to one example. In some examples, the delivery apparatus 100 is specifically adapted for use in introducing a prosthetic heart valve into a heart. For example, the delivery apparatus 100 can be used as the prosthetic valve delivery apparatus 60 in a prosthetic valve implantation procedure, as described above with reference to FIG. 3A.

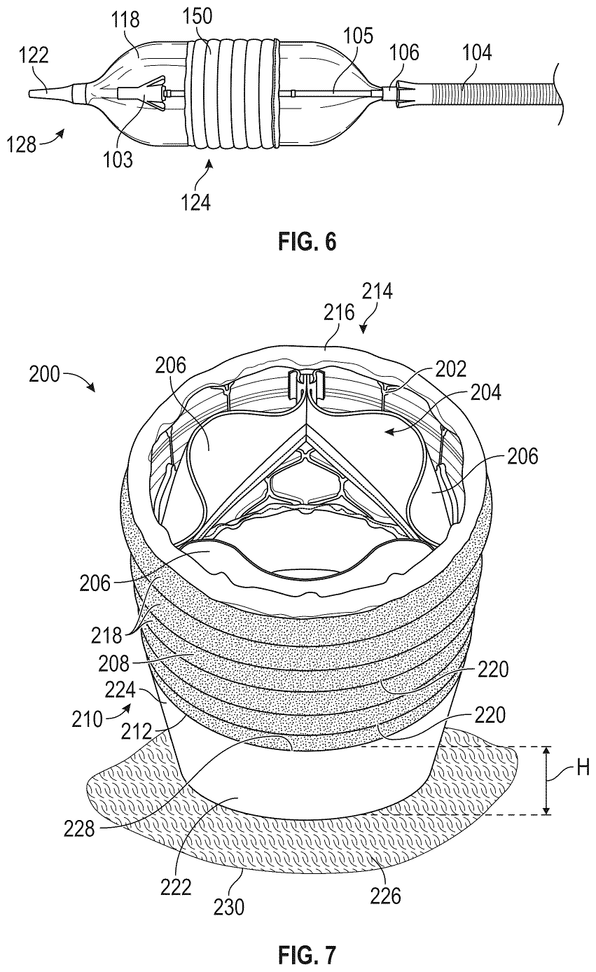

The delivery apparatus 100 in the illustrated example of FIG. 5 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. In some examples, the delivery apparatus 100 can further comprise an inner shaft 105 (FIG. 6) extending distally from the handle 102 coaxially through the intermediate shaft 106 and the outer shaft 104 and proximally from the handle 102 coaxially through the intermediate shaft.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion that extends proximally from a proximal end of the handle 102, to an adaptor 112. The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

In some examples, the intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 105 (FIG. 6) can extend distally beyond the distal end portion of the intermediate shaft 106 toward or to a nose cone 122 at a distal end of the delivery apparatus 100.

In some examples, a distal end of the balloon 118 can be coupled to a distal end of the delivery apparatus 100, such as to the nose cone 122 (as shown in FIG. 5), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder of the delivery apparatus 100. As shown in FIG. 5, a prosthetic heart valve 150 can be mounted around the balloon 118, at the valve mounting portion 124 of the delivery apparatus 100, in a radially compressed state. The prosthetic heart valve 150 can be configured to be radially expanded by inflation of the balloon 118 at a native valve annulus, as described above with reference to FIG. 3A.

A balloon shoulder assembly 103 (see FIG. 6) of the delivery apparatus 100, which includes the distal shoulder, is configured to maintain the prosthetic heart valve 150 (or other medical device) at a fixed position on the balloon 118 during delivery through the patient's vasculature.

The outer shaft 104 can include a distal tip portion 128 mounted on its distal end. In some examples, the outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 105 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 105 and an inner surface of the balloon 118. As such, fluid from the fluid source can flow to the fluid passageway from the annular space to inflate the balloon 118 and radially expand and deploy the prosthetic valve 150. FIG. 6 illustrates the balloon 118 and prosthetic valve 150 in the expanded configuration.

An inner lumen of the inner shaft 105 can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, incorporated by reference herein in its entirety.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site).

Prosthetic valves disclosed herein (e.g., prosthetic heart valve 150, prosthetic heart valve 62, etc.) can be radially compressible and expandable between a radially compressed state and a radially expanded state (see FIG. 6). Thus, the prosthetic valves can be crimped on or retained by an implant delivery apparatus (e.g., delivery apparatus 100, prosthetic valve delivery apparatus 60, etc.) in the radially compressed state during delivery, and then expanded to the radially expanded state once the prosthetic valve reaches the implantation site.

It is understood that the prosthetic valves disclosed herein may be used with a variety of implant delivery apparatuses and can be implanted via various delivery procedures, examples of which will be discussed in more detail later.

In some examples, in lieu of or in addition to the prosthetic valves 62 and/or 150 described herein, the prosthetic heart valve delivery apparatus (e.g., prosthetic heart valve delivery apparatuses 100 and/or 62 described herein) can be used to implant a prosthetic heart valve 200. Prosthetic valve 200 can be similar to prosthetic valves 62 and 150 described previously, however, prosthetic valve 200 can comprise a sealing member 222 configured to prevent or mitigate paravalvular leakage (PVL).

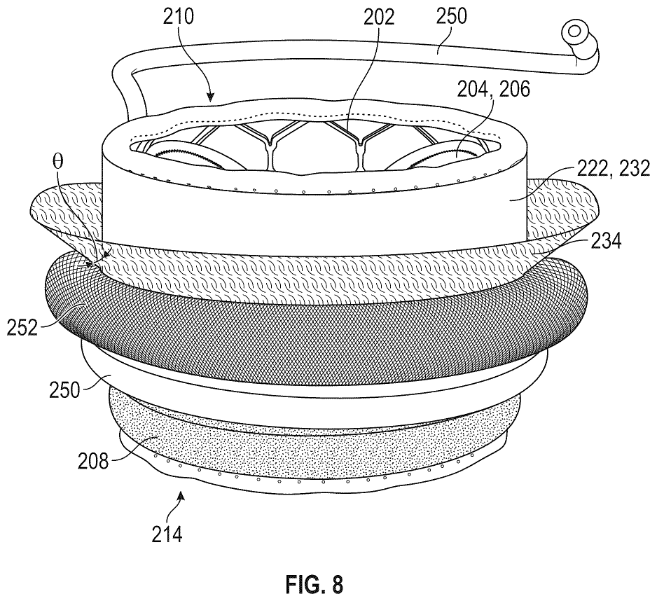

FIG. 7 illustrates the prosthetic valve 200 in a radially expanded configuration. The prosthetic valve 200 can be used as the prosthetic heart valve 62 in a prosthetic valve implantation procedure, as described above with reference to FIGS. 1-4. Any of the prosthetic valves disclosed herein are adapted to be implanted in the native mitral annulus or the native aortic annulus, although in other examples they can be adapted to be implanted in the other native annuluses of the heart (e.g., the pulmonary and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, as discussed previously, the prosthetic valve 200 can be implanted within a docking or anchoring device (e.g., docking device 52, etc.) that is implanted within a native heart valve or a vessel. For example, the disclosed prosthetic valves can be implanted within a docking device implanted within or at the native mitral valve, such as disclosed in U.S. Publication No. 2022/0079749, which is incorporated herein by reference in its entirety. In other examples the disclosed prosthetic valves can be implanted within, for example, a diseased pulmonary valve as disclosed in U.S. Pat. No. 10,363,130, which is incorporated by reference herein in its entirety, or within the superior or inferior vena cava for replacing the function of a diseased tricuspid valve, such as disclosed in U.S. Pat. No. 11,291,540, which is incorporated herein by reference in its entirety.

Referring to FIG. 7, the prosthetic valve 200 can include a radially expandable and compressible frame 202 and a valvular structure 204 comprising a plurality of leaflets 206. The valvular structure can be situated at least partially within the frame 202. In some examples, the prosthetic valve 200 can comprise an outer covering or outer skirt 208 disposed around an outer surface of the frame. The prosthetic valve can include an inflow end portion 210 terminating in an inflow edge 212 and an outflow end portion 214 terminating in an outflow edge 216. The terms “inflow” and “outflow” are related to the normal direction of blood flow (e.g., antegrade blood flow) through the prosthetic valve 200. For example, the leaflets 206 can allow blood flow through the valve 200 in a direction from the inflow end 210 to the outflow end 214 and prevent the reverse flow (e.g., prevent flow in a direction from the outflow end 214 to the inflow end 210).

The frame 202 can be made of any of various suitable plastically-expandable materials including include, metal alloys, polymers, or combinations thereof. Example metal alloys can comprise one or more of the following: nickel, cobalt, chromium, molybdenum, titanium, or other biocompatible metal. In some examples, the frame 202 can comprise stainless steel. In some examples, the frame 202 can comprise cobalt-chromium. In some examples, the frame 202 can comprise nickel-cobalt-chromium. In some examples, the frame 202 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. In other examples, the frame can comprise a self-expanding material (e.g., Nitinol) as known in the art.

When constructed of a plastically-expandable material, the frame 202 (and thus the valve 200) can be crimped to a radially compressed state on a delivery catheter, see e.g., prosthetic valve 150 crimped to delivery apparatus 100 in FIG. 5, and then expanded inside a patient by an inflatable balloon or equivalent expansion mechanism, see e.g., prosthetic valve 150 disposed in a radially expanded configuration on expanded balloon 118 in FIG. 6. When constructed of a self-expandable material, the 202 (and thus the valve 200) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

The outer skirt 208 can be wholly or partly formed of any suitable biological material, synthetic material (for example, any of various polymers), or combinations thereof. In some examples, the outer skirt 208 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the outer skirt 208 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the outer skirt 208 can comprise a non-textile or non-fabric material, such as a film including any of a variety of crystalline or semi-crystalline polymeric materials, such as PTFE, PET, polypropylene, polyamide, polyetheretherketone (PEEK), etc. In some examples, the outer covering 208 can comprise a sponge material or foam, such as polyurethane foam. In some examples, the outer covering 208 can comprise natural tissue, such as pericardium (for example, bovine pericardium, porcine pericardium, equine pericardium, or pericardium from other sources).

Further details of the prosthetic heart valve and its variants are described in U.S. Pat. Nos. 11,185,406 and 11,654,023 and U.S. Publication No. 2022/0079749, which are incorporated by reference herein in their entirety.

In some examples, such as shown in FIG. 7, the outer skirt 208 can comprise a plurality of floating yarn sections 218 (e.g., protruding or puffing sections) separated by a plurality of circumferentially-extending bands 220. In some examples, the bands 220 can be constructed via a leno weave, which improves the strength of the woven structure. In some examples of woven cloth, vertical fibers (e.g., running along the longitudinal axis of the valve 200) comprise a yarn or other fiber possessing a high level of expansion, such as a texturized weft yam, while horizontal (e.g., running circumferentially around valve 200) fibers in a leno weave comprise a low expansion yam or fiber. In some examples, the outer skirt 208 can extend from the inflow end portion 210 to the outflow end portion 214 of the prosthetic valve 200. In other examples, the outer skirt 208 can extend a distance less than a full axial distance of the prosthetic valve. Further details of the outer skirt 208 can be found, at least, in U.S. Pat. No. 11,654,023 and U.S. Publication No. 2022/0079749.

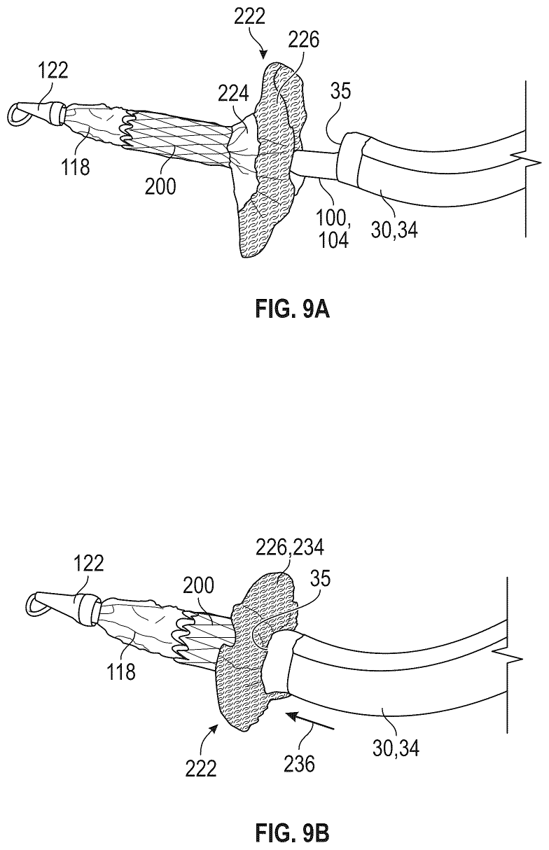

Referring still to FIG. 7, as mentioned previously, the prosthetic valve 200 can comprise a sealing member 222. The sealing member 222 can be used in lieu of or in addition to the outer skirt 208 for establishing a seal against the surrounding anatomy once implanted. For example, FIGS. 7 and 8 show a prosthetic valve 200 that includes both an outer skirt 208 and a sealing member 222. The sealing member 222 can be configured to fully or partially evert over the inflow end portion 210 of the prosthetic valve 200 in order to supplement sealing of the prosthetic valve at, for example, the lateral mitral commissure. FIG. 7 shows the sealing member 222 in a delivery configuration, and FIG. 8 shows the sealing member 222 in a partially everted position, also referred to as a deployed configuration.

Referring to FIG. 7, the sealing member 222 can comprise a first end portion 224 and a second end portion 226. The first end portion 224 can be coupled to the inflow end portion 210 of the prosthetic valve 200, e.g., using sutures. For example, in instances where the prosthetic valve also comprises an outer skirt 208, a first edge 228 of the sealing member 222 can be sutured to the inflow end portion of the outer skirt 208. In instances where the prosthetic valve 200 does not also comprise an outer skirt 208, the inflow edge 228 of the sealing member 222 can be secured to either the outer or inner surface of the frame. In examples where the sealing member 222 is secured to the inner surface of the frame 202, when in the deployed configuration, the sealing member 222 can wrap around the inflow edge 212 of the prosthetic valve 200. The sealing member 222 can be secured to an outer surface of the frame 202, an inner surface of the frame 202, an inner skirt (not shown), or to itself through the one or more openings in the frame 202. Also, the first end portion 224 need not be connected to the inflow end portion 210 of the prosthetic valve and instead can connected to the prosthetic valve (e.g., to the frame and/or to another skirt) at a location along the outflow end portion 214 or a location between the inflow end portion 210 and the outflow end portion 214.

In some examples, such as the illustrated example, when in the delivery configuration the first end portion 224 can have a first diameter and the second end portion 226 can have a second, greater diameter, or vice versa. In some examples, when in the delivery configuration the sealing member 222 can have a cylindrical shape wherein the first end portion 224 has a diameter substantially the same as a diameter of the second end portion 226. During implantation of the prosthetic valve 200, a portion of a delivery apparatus, (e.g., a portion of the guide catheter shaft 34) can be used to partially evert the sealing member 222 into the deployed configuration over the inflow end of the prosthetic valve 200. The prosthetic valve 200 can then be radially expanded to ‘lock’ the sealing member 222 in the deployed configuration.

In some examples, such as examples where the second end portion 226 has a diameter substantially the same as the first end portion 224, the second end portion 226 can comprise one or more slits or cuts extending from a second edge 230 partially along the axial length of the sealing member 222. The slits can allow the second end portion 226 to flare radially outwardly when the sealing member 222 is in the deployed configuration.

In some examples, the sealing member 222 can comprise polyethylene terephthalate (PET). The first end portion 224 of the sealing member 222 can comprise, for example, a thin, woven PET ribbon fabric or cloth. The second end portion 226 can comprise, for example, a PET knitted fabric or cloth having a thickness greater than a thickness of the first end portion 224. The thin material comprising the first end portion 224 allows the first end portion 224 to be quickly and easily everted over the inflow end 210 of the prosthetic valve 200 during deployment of the prosthetic valve. The thicker material of the second end portion 226 provides structure that allows the second end portion 226 to form the flanged portion 234 when in the deployed configuration.

As shown in FIG. 7, when in the delivery configuration, the first portion 224 of the sealing member 222 can have an annular shape having a height H extending from the inflow end portion 210 of the prosthetic valve 200. The second end portion 226 can extend from the first end portion 224 and can comprise a frustoconical or flared shape. Referring to FIG. 8, when in the deployed or partially everted configuration, the first portion 224 can be configured as an axially-extending portion 232 that extends partially over the inflow end portion 210 of the prosthetic valve 200 and the second portion 226 of the sealing member can be configured as a flared or flanged portion 234 that flares laterally from a surface of the frame. In some examples, the flanged portion 234 can be shape set to flare radially outwardly once everted. In some examples, the flanged portion 234 can comprise one or more ribs, wires, or other structural support members to maintain the flanged portion 234 in the everted position.

In some examples, the axially-extending portion 232 can have substantially the same axial length as the flanged portion 234. In other examples, the axially-extending portion 232 can be axially longer than the flanged portion 234, or vice versa.

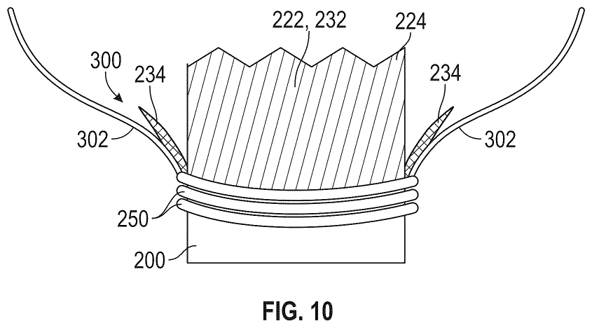

In some examples, the flanged portion 234 can extend from the radial outer surface of the prosthetic valve laterally at a non-perpendicular angle. For example, as shown in FIG. 8, the flanged portion can extend at an angle θ back toward the inflow end portion 210 of the prosthetic valve 200 such that the flanged portion 234 fully or partially overlaps the axially-extending portion 232. The angle θ can substantially correspond to the slope of the native mitral valve such that the flanged portion 234 can be seated against the walls 302 of the native mitral valve 300, as shown in FIG. 10. In other examples, the flanged portion 234 can extend laterally from the radial outside of the prosthetic valve 200 at a perpendicular or substantially perpendicular angle. In some examples, the flanged portion 234 can have a width between about 7 mm and about 9 mm.

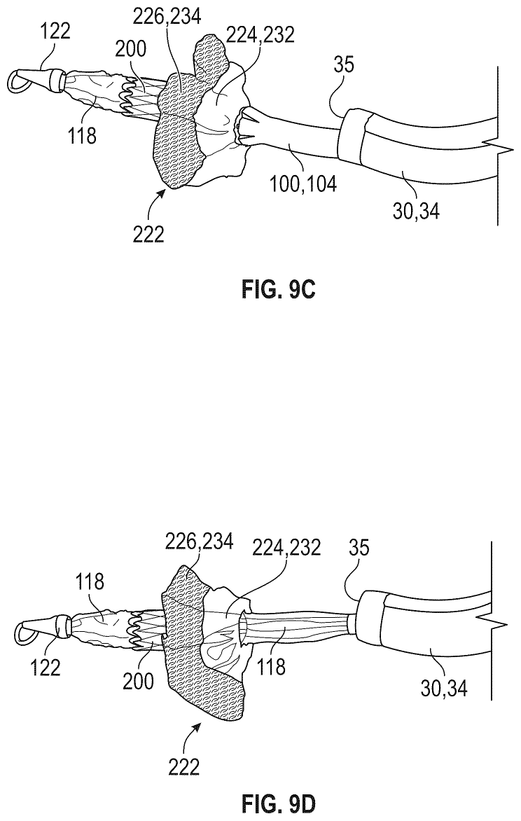

As shown in FIG. 8, the prosthetic valve 200 can be seated within a docking device, such as docking device 250. Docking device 250 can be the same as or similar to docking device 52 described previously. In some examples, docking device 250 can comprise a guard member 252 coupled to the docking device 250. The guard member 252 can be configured to prevent or mitigate paravalvular leakage between the annulus of the native mitral valve 300 and a prosthetic heart valve (such as the prosthetic heart valve 200) positioned in the docking device 250. In some examples, the sealing member 222 can function cooperatively with the guard member 252. In other examples, such as shown in FIGS. 9-10, the sealing member 222 can be used in lieu of the guard member 252. Further details of the docking device 250 and guard member 252 can be found, at least, in U.S. Provisional App. No. 63/488,511.

The prosthetic valve 200 can be implanted within a docking device, such as docking device 250, using a delivery apparatus, such as delivery apparatus 100 described previously, according to the following exemplary method. The distal end portion of a guide catheter 30 (comprising a handle 32 and a catheter shaft 34) can be inserted into a patient as described previously with reference to FIGS. 1-4, and the docking device 250 can be inserted and implanted through the guide catheter 30 in the manner described previously with reference to FIGS. 1-2B.

The prosthetic valve 200 can then be mounted in a radially compressed state along a distal end portion 128 of the delivery apparatus 100 such that the sealing member 222 extends from the inflow end portion 210 of the prosthetic valve 200 in a proximal direction over a distal end portion of the outer shaft 104 of the delivery apparatus (see FIG. 9A, for example). Such a configuration advantageously allows the addition of the sealing member 222 without affecting the crimp profile of the prosthetic valve 200 during delivery since the sealing member 222 is disposed almost entirely off of the prosthetic valve 200 when in the delivery configuration.

The distal end of the delivery apparatus 100 (including the prosthetic valve 200) can then be advanced through the guide catheter shaft 34 in the manner described previously with respect to FIG. 3A. Referring to FIGS. 9A-9D, once the prosthetic valve 200 is positioned adjacent a desired implantation site, such as, e.g., adjacent a docking device 250 within the patient's native mitral valve 300, the guide catheter shaft 34 can be advanced distally as indicated by arrow 236 (FIG. 9B) such that a distal edge 35 of the guide catheter shaft 34 contacts the second end portion 226 of the sealing member 222 and applies force to the sealing member 222 to evert the first end portion 224 of the sealing member 222 over the inflow end portion 210 of the prosthetic valve 200. The guide catheter shaft 34 can then be retracted proximally as shown in FIG. 9C, leaving the first end portion 224 everted over the inflow end portion of the prosthetic valve. The outer shaft 104 can then be retracted proximally as shown in FIG. 9D, allowing the balloon 118 to be inflated.

Once the sealing member 222 is in the deployed configuration (i.e., the partially everted or everted configuration), the prosthetic valve 200 can be further advanced into the docking device 250 (if not already disposed within the docking device 250) and radially expanded by inflating the balloon 118 to seat the prosthetic valve 200 within the docking device 250 (such as shown in FIG. 8) and ‘lock’ the flanged portion in the deployed configuration. As shown in FIG. 10, the prosthetic valve 200 can be seated within the docking device 250 such that the flanged portion 234 is upstream of the docking device 250. The flanged portion 234 can thus sit flush or substantially flush against the inflow surfaces of the native mitral valve leaflets 302, thereby preventing or mitigating PVL at the lateral commissure.

Such a configuration advantageously allows 360 degrees of PVL prevention and/or mitigation at the mitral valve. Disposing the sealing member 222 on the delivery apparatus 100 when in the delivery configuration advantageously allows the sealing member 222 to be added without affecting the overall crimp profile of the prosthetic valve 200. Further, positioning the sealing member 222 on the shaft 104 of the delivery apparatus 100 allows for a lower push force required to partially evert the sealing member 222 into the deployed configuration. In some examples, the force required can be less than 15 N.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. An implantable prosthetic valve, comprising:
a radially expandable and compressible annular frame comprising an inflow end portion and an outflow end portion;
a valvular structure comprising a plurality of leaflets arranged within the frame;
a sealing member having a first end portion and a second end portion, the first end portion being coupled to an inflow edge of the frame, the sealing member being movable between a delivery configuration wherein the first end portion extends proximally from the frame and a deployed configuration wherein the first end portion is everted over the inflow end of the frame such that the second end portion forms a flanged portion.
Example 2. The prosthetic valve of any example herein, particularly example 1, further comprising an outer skirt disposed about an outer circumference of the frame.
Example 3. The prosthetic valve of any example herein, particularly example 2, wherein the first end portion of the sealing member is coupled to an inflow end portion of the outer skirt.
Example 4. The prosthetic valve of any example herein, particularly any one of examples 1-3, wherein when in the deployed configuration the sealing member comprises an axially extending portion disposed adjacent an outer surface of the frame and the flanged portion extending laterally from the outer surface of the frame.
Example 5. The prosthetic valve of any example herein, particularly example 4, wherein the flanged portion extends at a non-perpendicular angle relative to the outer surface of the frame.
Example 6. The prosthetic valve of any example herein, particularly example 4, wherein the flanged portion extends toward the inflow end portion of the frame such that the flanged portion at least partially overlaps the axially extending portion.
Example 7. The prosthetic valve of any example herein, particularly any one of examples 1-6, wherein the first end portion is coupled to an outer surface of the prosthetic valve.
Example 8. The prosthetic valve of any example herein, particularly any one of examples 1-6, wherein the first end portion is coupled to an inner surface of the prosthetic valve such that when the sealing member is in the deployed position the sealing member extends over an inflow edge of the frame.
Example 9. The prosthetic valve of any example herein, particularly any one of examples 1-8, wherein the second end portion of the sealing member comprises one or more slits configured to allow the second end portion to flare laterally outwardly when in the deployed configuration.
Example 10. The prosthetic valve of any example herein, particularly any one of examples 1-9, wherein the second end portion of the sealing member is shape set such that the second end portion flares laterally outwardly when in the deployed configuration.
Example 11. The prosthetic valve of any example herein, particularly any one of examples 1-10, wherein the second end portion of the sealing member comprises one or more ribs.
Example 12. The prosthetic valve of any example herein, particularly any one of examples 1-11, wherein the flanged portion extends at a perpendicular angle relative to the outer surface of the frame.
Example 13. The prosthetic valve of any example herein, particularly any one of examples 1-12, wherein the sealing member has a cylindrical shape wherein the first end portion and the second end portion have substantially the same diameter.
Example 14. The prosthetic valve of any example herein, particularly any one of examples 1-12, wherein the sealing member has a frustoconical shape wherein the first end portion has a diameter smaller than the second end portion.
Example 15. An assembly, comprising:
a guide catheter including a guide catheter shaft;
a prosthetic valve delivery apparatus comprising:
   a handle,
   a delivery shaft extending from a distal end portion of the handle and comprising a delivery shaft lumen, and
   an inflatable member coupled to a distal end portion of the delivery shaft; and
a radially expandable and compressible implantable prosthetic device disposed over the inflatable member, the prosthetic device comprising:
   an annular frame comprising an inflow end portion and an outflow end portion,
   a valvular structure comprising a plurality of leaflets arranged within the frame, and
   a sealing member having a first end portion and a second end portion, the first end portion being coupled to an inflow edge of the frame, the sealing member being movable between a delivery configuration and a deployed configuration;
wherein when the sealing member is in the delivery configuration the sealing member extends proximally over the delivery shaft of the prosthetic valve delivery apparatus.
Example 16. The assembly of any example herein, particularly example 15, wherein the guide catheter shaft is configured to move the sealing member from the delivery configuration to the deployed configuration.
Example 17. The assembly of any example herein, particularly any one of examples 15-16, wherein when in the deployed configuration the sealing member comprises an axially extending portion disposed adjacent an outer surface of the frame and the flanged portion extending laterally from the outer surface of the frame.
Example 18. The assembly of any example herein, particularly example 17, wherein when in the deployed configuration the flanged portion extends at a non-perpendicular angle relative to the outer surface of the frame.
Example 19. The assembly of any example herein, particularly any one of examples 17-18, wherein the flanged portion extends toward the inflow end portion of the frame such that the flanged portion at least partially overlaps the axially extending portion.
Example 20. The assembly of any example herein, particularly any one of examples 15-19, wherein the guide catheter shaft is configured to selectively move the sealing member from the delivery configuration to the deployed configuration.
Example 21. The assembly of any example herein, particularly any one of examples 15-20, further comprising an outer skirt disposed about an outer circumference of the frame.
Example 22. The assembly of any example herein, particularly any one of examples 15-21, wherein the second end portion of the sealing member comprises one or more slits configured to allow the second end portion to flare laterally outwardly when in the deployed configuration.
Example 23. The assembly of any example herein, particularly any one of examples 15-22, wherein the second end portion of the sealing member is shape set such that the second end portion flares laterally outwardly when in the deployed configuration.
Example 24. The assembly of any example herein, particularly any one of examples 15-23, wherein the second end portion of the sealing member comprises one or more ribs.
Example 25. The prosthetic valve of any example herein, particularly any one of examples 15-25, wherein when in the deployed configuration the sealing member comprises a flanged portion and wherein the flanged portion extends at a perpendicular angle relative to the outer surface of the frame.
Example 26. The prosthetic valve of any example herein, particularly any one of examples 15-25 wherein the sealing member has a cylindrical shape wherein the first end portion and the second end portion have substantially the same diameter.
Example 27 The prosthetic valve of any example herein, particularly any one of examples 15-25, wherein the sealing member has a frustoconical shape wherein the first end portion has a diameter smaller than the second end portion.
Example 28. A method of delivering an implantable prosthetic valve to a native valve of a heart, comprising:
advancing a distal end portion of a delivery apparatus through a guide catheter to a selected implantation site, the distal end portion of the delivery apparatus comprising a prosthetic valve disposed over an inflatable member, the prosthetic valve comprising a radially expandable and compressible annular frame, a valvular structure comprising a plurality of leaflets arranged within the frame, and a sealing member having a first end portion and a second end portion, the first end portion being coupled to an inflow edge of the frame and the second end portion being disposed over a shaft of the delivery apparatus;
using a distal end portion of the guide catheter shaft to partially evert the sealing member over an inflow end portion of the prosthetic valve such that the first end portion of the sealing member is disposed adjacent an outer surface of the prosthetic valve and such that the second end portion of the sealing member forms a flanged portion extending laterally outwardly from an outer surface of the prosthetic valve;
positioning the prosthetic valve at the selected implantation site; and
radially expanding the prosthetic valve to seat the prosthetic valve at the selected implantation site.
Example 29. The method of any example herein, particularly example 28, wherein using a distal end portion of the guide catheter to partially evert the sealing member comprises positioning a distal edge of the guide catheter such that it abuts the second end portion of the sealing member and advancing the guide catheter to apply a force to the second end portion of the sealing member such that it everts over the first end portion of the sealing member.
Example 30. The method of any example herein, particularly any one of examples 28-29, wherein when the prosthetic valve is seated at the selected implantation site the flanged portion sits against the walls of the selected implantation site to mitigate PVL.
Example 31. The method of any example herein, particularly any one of examples 28-30, wherein the selected implantation site is a native mitral valve.
Example 32. The method of any example herein, particularly any one of examples 28-31, wherein the selected implantation site is within a previously deployed docking device disposed at a native mitral valve.
Example 33. A prosthetic heart valve according to any example herein, wherein the prosthetic heart valve is sterilized.
Example 34. A method of sterilizing any of the prosthetic heart valves described herein.

The features described herein with regard to any example can be combined with other features described in any one or more of the other examples, unless otherwise stated. For example, any one or more of the features of one sealing member can be combined with any one or more features of another sealing member or another prosthetic valve. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. An implantable prosthetic valve, comprising:
a radially expandable and compressible annular frame comprising an inflow end portion and an outflow end portion;
a valvular structure comprising a plurality of leaflets arranged within the frame; and
a sealing member having a first end portion and a second end portion, the first end portion being coupled to an inflow edge of the frame, the sealing member being movable between a delivery configuration wherein the first end portion extends proximally from the frame and a deployed configuration wherein the first end portion is everted over the inflow end of the frame such that the second end portion forms a flanged portion.

2. The prosthetic valve of claim 1, further comprising an outer skirt disposed around an outer circumference of the frame.

3. The prosthetic valve of claim 2, wherein the first end portion of the sealing member is coupled to an inflow end portion of the outer skirt.

4. The prosthetic valve of claim 1, wherein when in the deployed configuration the sealing member comprises an axially extending portion disposed adjacent an outer surface of the frame and the flanged portion extending laterally from the outer surface of the frame.

5. The prosthetic valve of claim 4, wherein the flanged portion extends at a non-perpendicular angle relative to the outer surface of the frame.

6. The prosthetic valve of claim 4, wherein the flanged portion extends toward the inflow end portion of the frame such that the flanged portion at least partially overlaps the axially extending portion.

7. The prosthetic valve of claim 1, wherein the first end portion is coupled to an outer surface of the prosthetic valve.

8. The prosthetic valve of claim 1, wherein the first end portion is coupled to an inner surface of the prosthetic valve such that when the sealing member is in the deployed position the sealing member extends over an inflow edge of the frame.

9. The prosthetic valve of claim 1, wherein the second end portion of the sealing member is shape set such that the second end portion flares laterally outwardly when in the deployed configuration.

10. An assembly, comprising:
a guide catheter including a guide catheter shaft;
a prosthetic valve delivery apparatus comprising:
a handle,
a delivery shaft extending from a distal end portion of the handle and comprising a delivery shaft lumen, and
an inflatable member coupled to a distal end portion of the delivery shaft; and
a radially expandable and compressible implantable prosthetic valve disposed over the inflatable member in a radially compressed state, the prosthetic device comprising:
an annular frame comprising an inflow end portion and an outflow end portion,
a valvular structure comprising a plurality of leaflets arranged within the frame, and
a sealing member having a first end portion and a second end portion the first end portion being coupled to an inflow edge of the frame, the sealing member being movable between a delivery configuration and a deployed configuration; and
wherein when the sealing member is in the delivery configuration the sealing member extends proximally over the delivery shaft of the prosthetic valve delivery apparatus.

11. The assembly of claim 10, wherein the guide catheter shaft is configured to move the sealing member from the delivery configuration to the deployed configuration.

12. The assembly of claim 10, wherein when in the deployed configuration the sealing member comprises an axially extending portion disposed adjacent an outer surface of the frame and a flanged portion extending laterally from the outer surface of the frame.

13. The assembly of claim 12, wherein when in the deployed configuration the flanged portion extends at a non-perpendicular angle relative to the outer surface of the frame.

14. The assembly of claim 12, wherein the flanged portion extends toward the inflow end portion of the frame such that the flanged portion at least partially overlaps the axially extending portion.

15. The assembly of claim 10, further comprising an outer skirt disposed around an outer circumference of the frame.

16. A method of delivering an implantable prosthetic valve to a native valve of a heart, comprising:
advancing a distal end portion of a delivery apparatus through a guide catheter shaft to a selected implantation site, the distal end portion of the delivery apparatus comprising a prosthetic valve disposed over an inflatable member, the prosthetic valve comprising a radially expandable and compressible annular frame, a valvular structure comprising a plurality of leaflets arranged within the frame, and a sealing member having a first end portion and a second end portion, the first end portion being coupled to an inflow edge of the frame and the second end portion being disposed over a shaft of the delivery apparatus;
using a distal end portion of the guide catheter shaft to partially evert the sealing member over an inflow end portion of the prosthetic valve such that the first end portion of the sealing member is disposed adjacent an outer surface of the prosthetic valve and such that the second end portion of the sealing member forms a flanged portion extending laterally outwardly from an outer surface of the prosthetic valve;
positioning the prosthetic valve at the selected implantation site; and
radially expanding the prosthetic valve to seat the prosthetic valve at the selected implantation site.

17. The method of claim 16, wherein using a distal end portion of the guide catheter shaft to partially evert the sealing member comprises positioning a distal edge of the guide catheter shaft such that it abuts the second end portion of the sealing member and advancing the guide catheter shaft to apply a force to the second end portion of the sealing member such that it everts over the first end portion of the sealing member.

18. The method of claim 16, wherein when the prosthetic valve is seated at the selected implantation site the flanged portion sits against one or more native walls of the selected implantation site to mitigate PVL.

19. The method of claim 16, wherein the selected implantation site is a native mitral valve.

20. The method of claim 16, wherein the selected implantation site is within a previously deployed docking device disposed at a native mitral valve.
